(19) United States
(12) Patent Application Publication
(10) Pub. No.: US 2018/0021728 A1
(43) Pub. Date: Jan. 25, 2018

(54) Pervaporation and Vapor-Permeation Separation of Gas-Liquid Mixtures and Liquid Mistures by SAPO-34 Molecular Sieve Membrane Prepared in Dry-Gel Process

(71) Applicants: Shanghai Advanced Research Institute, Chinese Academy of Sciences, Shanghai (CN); Shanxi Lu'An Enviromental Protection & Energy Development Co., Ltd., Changzhi (CN); Total Raffinage Chimie, Courbevoie (FR)

(72) Inventors: Daniel Curulla-Ferre, Uccle (BE); Yuhan Sun, Shanghai (CN); Zhiqiang Sun, Changzhi (CN); Yanfeng Zhang, Shanghai (CN)

(73) Assignee: Shanghai Advanced Research Institute, Chinese Academy of Sciences, Shanghai (CN)

(21) Appl. No.: 15/547,923

(22) PCT Filed: Feb. 2, 2016
(86) PCT No.: PCT/EP2016/052208

§ 371 Date: Aug. 1, 2017

(30) Foreign Application Priority Data
Feb. 3, 2015 (CN) 201510054314.4

(51) Int. Cl. B01D 61/36; B01D 71/02; C01B 39/54; B01D 69/10; C07C 68/08; B01D 19/00; B01D 67/00

(52) U.S. Cl. CPC B01D 61/362; B01D 19/0031; B01D 71/028; B01D 67/0051; B01D 69/105; C07C 68/08; C01B 39/54; B01D 2323/46

(57) ABSTRACT

The invention discloses a method for the pervaporation and vapor-permeation separation of a gas-liquid mixture or a liquid mixture by a SAPO-34 molecular sieve membrane prepared in a dry gel process, comprising: 1) synthesis of SAPO-34 molecular sieve seeds; 2) coating the SAPO-34 seeds on a porous support; 3) preparation of a mother liquor for dry gel synthesis of SAPO-34 molecular sieve membrane; 4) supporting the mother liquor for dry gel synthesis on the porous support coated with SAPO molecular sieve seeds and drying; 5) placing the porous support prepared in step 4) into a reaction vessel, adding a solvent, performing crystallization of the dry gel; 6) calcining; 7) using the SAPO-34 molecular sieve membrane obtained from step 6) to perform separation of a gas-liquid mixture or a liquid mixture by a process of pervaporation separation or vapor-permeation separation. The invention has the advantages of very high methanol selectivity and permeation flux, lowering synthesis cost of molecular sieve membrane and lowering environment pollution.

### FIELD OF THE INVENTION

The invention relates to a method for separation of a mixture by a SAPO-34 molecular sieve membrane, especially a method for the pervaporation (pervaporative separation) and vapor-permeation separation of a gas-liquid mixture or a liquid mixture by a SAPO-34 molecular sieve membrane prepared in a dry-gel process.

### BACKGROUND OF THE INVENTION

Dimethyl carbonate (DMC), which has a molecular formula of CO(OCH₃)₂, is a good solvent, has low volatility and similar toxicity values to anhydrous ethanol, and is completely biodegradable. It is an environmental-friendly chemical; its molecules have an oxygen content of 53%, which is three times higher than that of methyl tert-butyl ether (MTBE). It can be used as an additive in gasoline to enhance octane number and to suppress emission of carbon monoxide and hydrocarbons. It is very active in terms of chemistry, and it is an important intermediate and starting material for organic synthesis. Dimethyl carbonate finds extensive applications in the fields of pharmaceutical, chemical engineering and energy etc, and is receiving increasing attention. It has been rapidly developed and it is known as a new foundation of organic synthesis.

The industrial methods for producing DMC mainly include methods of oxidative carbonylation, transesterification, or phosgenation of methanol [Applied Catalysis A: General, 221(2001) 241-251]. No matter which method is used, a mixture of methanol (MeOH) and DMC was always obtained from the reaction. At normal pressure, MeOH and DMC would form a binary azeotrope (70 wt % MeOH and 30 wt % DMC), whose azeotropic temperature is 64° C. Therefore, it is a necessary to separate and recover DMC from the azeotrope. Currently, methods for separation of the MeOH/DMC azeotrope mainly include low temperature crystallization, adsorption, extractive distillation, azeotropic distillation and pressure distillation. All of these methods possess the disadvantages and shortcomings that energy consumption is high, it is difficult to select the appropriate solvent, it is difficult to operate and the safety has deficiencies. In contrast, a pervaporation method possesses advantages of low energy consumption, high efficiency and flexible operation conditions.

The pervaporation is a new membrane technology for separation. It uses the differential chemical potentials of a component on both sides of the membrane as a driving force. The membrane can be used to achieve selective separation of different components in feed liquids according to different affinity and mass transfer resistance of the components. Currently, the membranes used for pervaporation mainly include polymeric membrane, inorganic membrane and composite membrane. Rencetly, some progress has been made in studies on pervaporation separation of MeOH/DMC mixtures. Most of the studies focused on the polymeric membranes. The researchers found that materials such as polyvinyl alcohol (PVA), polyacrylic acid, chitosan or the like can be prepared into pervaporation membranes which preferentially remove methanol and have good separation performance.

Wooyoung et al. used a cross-linked chitosan membrane for pervaporation separation of MeOH/DMC and investigated the influences of operation temperature and feed composition on the separation factor and flux and received a good result [Separation and Purification Technology 31 (2003) 129-140]. Wang et al. prepared a polyacrylic acid (PAA)/polyvinyl alcohol (PVA) mixed membrane, wherein a mixed membrane containing 70 wt % PPA has a separation factor of 13 and a permeation flux of 577 g/(m² h) [Journal of Membrane Science 305 (2007) 238-246]. Pasternak et al. tested the performance of a polyvinyl alcohol (PVA) membrane for the separation of MeOH/DMC; for a feed composition of 70/30 MeOH/DMC, a methanol solution of 93-97 wt % concentration is produced on the permeate side and the flux is 110-1130 g/(m² h) [U.S. Pat. No. 4,798,674 (1989)]. Chen et al. prepared a hybrid membrane of chitosan and silica through cross-linking chitosan with aminopropyl triethoxy silane. Separation factor of 30 and permeation flux of 1265 g/(m² h) were achieved at 50° C. for a 70/30 MeOH/DMC mixture.

The polymeric membranes have an advantage of low cost. However, they are also suffering from the disadvantages such as low chemical and thermal stability, easy to swell during the process of separation, and incapability of being used for separation at high pressure; all of which would influence the separation performance of the membranes. On the other hand, the inorganic membranes can well solve these issues because the inorganic membranes have a uniform pore size and high chemical and thermal stability. Therefore, the inorganic membranes can be used for the separation in an environment under harsh conditions and they are also suitable for separation under high pressure. Currently, the main application of inorganic zeolite molecular sieve membranes is dehydration of organics. Applications of molecular sieve membrane in the separation of MeOH/DMC were rarely reported. Li et al. prepared a ZSM-5 molecular sieve membrane on porous alumina support and used the same for the separation of a water/acetic acid mixture [Journal of Membrane Science 218 (2003) 185-194]. Pina et al. synthesized a NaA molecular sieve membrane on Al₂O₃ support and used the NaA molecular sieve membrane to separate a water/ethanol mixture by pervaporation, in which the separation factor can reach 3600 and the permeation flux of water reaches 3800 g/(m² h) [Journal of Membrane Science 244 (2004) 141-150]. Hidetoshi et al. prepared NaX and NaY membranes on supports and systemically studied the pervaporation separation performance of the membranes. It was found that the membranes have very high selectivity to alcohols and benzene. They also studied the selectivity of these membranes for MeOH/DMC separation, and as a result, separation factor of 480 and permeation flux of 1530 g/(m² h) were achieved while the feed composition was 50/50 [Separation and Purification Technology 25 (2001) 261-268].

### SUMMARY OF THE INVENTION

The technical problem to be solved by the present invention is to provide a method for the pervaporation and vapor-permeation separation of a gas-liquid mixture or a liquid mixture by a SAPO-34 molecular sieve membrane prepared in a dry gel process. The inventive method achieves very high methanol (MeOH) selectivity and permeation flux. It also reduces the cost of molecular sieve membrane synthesis and reduces environmental pollution.

To resolve the issues mentioned above, the invention provides a method for the pervaporation or vapor-permeation separation of a gas-liquid mixture or a liquid mixture (e.g., separation of a methanol-containing mixture) by a SAPO-34 molecular sieve membrane prepared in a dry gel process, which method includes the following steps:

1) synthesis of SAPO-34 molecular sieve seeds (crystal seeds)
mixing and dissolving an Al source, tetraethylammonium hydroxide (TEAOH, a template agent), water, a Si source and a P source to make a reaction liquor for seeds, which is then subjected to crystallization for 2-72 h by heating at 120-230° C., then centrifuging, washing and drying to get SAPO-34 molecular sieve seeds;
wherein the molar ratio of the Al source, P source, Si source, tetraethylammonium hydroxide and all water in the reaction liquor for seeds is 1 Al₂O₃: 1-2 P₂O₅: 0.3-0.6 SiO₂: 1-3 TEAOH: 55-150 H₂O;
2) coating (e.g., uniformly coating) the SAPO-34 molecular sieve seeds on a porous support;
3) preparation of a mother liquor for dry gel synthesis of SAPO-34 molecular sieve membrane uniformly mixing an Al source, a P source, a Si source, tetraethyl ammonium hydroxide (TEAOH) and water to form a mother liquor for dry gel synthesis of SAPO-34 molecular sieve membrane; wherein the molar ratio of the Al source, P source, Si source, tetraethyl ammonium hydroxide and all water in the mother liquor for dry gel synthesis is: 1 Al₂O₃: 1-2 P₂O₅: 0.1-0.6 SiO₂: 1-8 TEAOH:30-1000 H₂O;
4) supporting the mother liquor for dry gel synthesis on the porous support coated with SAPO-34 molecular sieve seeds prepared in step 2), and drying, to form a porous support having a dry gel layer;
5) placing the porous support prepared in step 4) into a reaction vessel, adding a solvent (no direct contact between the solvent and the membrane tube), performing crystallization of the dry gel;
6) calcining at a high temperature of 400-600° C. for 4-8 h for removal of the template agent, so as to obtain a SAPO-34 molecular sieve membrane;
7) using the SAPO-34 molecular sieve membrane obtained in step 6) to perform separation of a gas-liquid mixture or a liquid mixture by a process of pervaporation separation or vapor-permeation separation. The gas in the gas-liquid mixture includes common gases, for is selected from inert gas, hydrogen gas, oxygen gas, CO₂ or gaseous hydrocarbon, and the liquid in the gas-liquid mixture includes common solvents such as water, alcohol, ketone or aromatics;
wherein in the step 7), the inert gas contains N₂;
the gaseous hydrocarbon contains methane;
the alcohol contains methanol, ethanol, or propanol;
the ketone contains acetone or butanone;
the aromatics contain benzene.

In addition, in step 7), in the separation of the liquid mixtures by the SAPO-34 molecular sieve membrane, said liquid mixture is a mixture of methanol and a liquid other than methanol, said liquid other than methanol is selected from dimethyl carbonate, ethanol, methyl tert-butyl ether.

In the step 1), the detailed procedures for making the reaction liquor for seeds are: adding the Al source to the tetraethyl ammonium hydroxide solution, and after full hydrolysis, adding the Si source and P source, and stirring for 12-24 h to form the reaction liquor for seeds.

In the steps 1) and 3), the Al source is selected from one or more of aluminium isopropoxide, aluminium hydroxide, elemental aluminium, an aluminium salt, alumina, and hydrated alumina; wherein the aluminium salt is selected from one or more of aluminium nitrate, aluminium chloride, alumimium sulfate, aluminium phosphate; the Si source is selectged from one or more of silica sol, silicate ester, silica aerosol and sodium silicate; and the P source is phosphoric acid.

In the step 1), the heating is preferably microwave heating.

In step 1), the size of the SAPO-34 molecular sieve seeds is 50-1000 nm.

In the step 2), the shapes of the porous support are selected from single-channel tube, multi-channel tube, flat plate, hollow fiber tube; the material of the porous support is selected from ceramics, stainless steel, alumina, titania, zirconia, silica, silicon carbide, or silicon nitride; the pore size of the porous support is 5-2000 nm.

In the step 2), the coating method is selected from brush coating, dip-coating, spray coating or spin coating; wherein the procedure in case of dip-coating is: uniformly coating a 0.01-1 wt % (mass percentage) solution of SAPO-34 molecular sieve seeds in ethanol on the porous support.

In the step 3), the specific procedure of forming the mother liquor for dry gel synthesis of SAPO-34 molecular sieve membrane comprises: adding the Al source to the P source and after full hydrolysis, adding the Si source and tetraethyl ammonium hydroxide and stirring for 12-24 h, to obtain the mother liquor for dry gel synthesis of SAPO-34 molecular sieve membrane.

In the step 4), the supporting method is selected from dip-coating, spray coating or spin coating; wherein the specific procedure in the case of dip-coating comprises: immersing the porous support coated with SAPO-34 molecular sieve seeds in the mother liquor for dry gel synthesis for 1 s to 24 h, and then drying for 1 min to 48 h at room temperature to 120° C.

In the step 4), the molar composition of the dry gel layer is preferably 1 Al₂O₃: 1-2 P₂O₅: 0.1-0.6 SiO₂: 1-8 TEAOH: 1-300 H₂O.

In the step 5), the solvent is selected from one or more of water, ammonia water, the mother liquor for dry gel synthesis, an organic solvent; the solvent is used in an amount of 0.001-0.1 g per mL volume of the reaction vessel; moreover, in step 5), when in liquid state, the solvent does not directly contact the dry gel layer, but when the crystallization is performed at high temperature, the vapor produced after the vaporization of the solvent comes into direct contact with the dry gel layer.

In the step 5), the temperature for crystallization of the dry gel is 120-230° C. and the crystallization time is 2-72 h, preferably 4-7 h.

In the step 6), the atmosphere for calcination is selected from inert gas, vacuum, air, oxygen gas, or diluted oxygen gas in any ratio; and in the calcination, the temperature increasing rate and the temperature decreasing rate is not higher than 2 K/min.

In the step 6) the membrane thickness of the SAPO-34 molecular sieve membrane is 1-2 microns.

In the step 7), the conditions for the process of pervaporation separation or vapor-permeation separation are as follows: the methanol concentration of the feed is 1-99 wt %, the feed flow rate is 1-500 mL/min, the separation operation temperature is room temperature to 150° C., and the pressure on the permeate side is 0.06 to 300 Pa.

The invention provides a ultrathin SAPO-34 molecular sieve membrane in a dry gel process. Said membrane has a high flux, small thickness (can be as low as about 1 micron), so that the mass transfer resistance is low, and the permeation rate is high; when used for pervaporation separation of a gas-liquid mixture or a liquid mixture such as a MeOH/DMC mixture, it shows high MeOH selectivity and permeation flux. For example, when the SAPO-34 molecular sieve membrane is used for separation of a methanol/dimethyl carbonate mixture (in a mass ratio of 90/10) at a temperature of 120° C., the permeation flux reaches 10 kg·m⁻²·h⁻¹, the separation factor is above 500 and the methanol concentration on the permeate side is above 99.98 wt %.

On the other hand, in terms of the preparation of the molecular sieve membrane, the present invention greatly reduces consumption of starting materials and template agent for the synthesis and thus reduces the synthesis cost and reduces the environmental pollution. Furthermore, the prepared SAPO-34 molecular sieve membrane is very thin in thickness, thereby the mass transfer resistance is greatly decreased and the methanol flux through the membrane is improved. Therefore, the invention will be of great importance in industrial application because it provides a high-efficiency, environmental-friendly and economic method for separation of MeOH/DMC mixture

In addition to the separation of a methanol/dimethyl carbonate mixture, the SAPO-34 molecular sieve membrane of the present invention can also be applied for the pervaporation or vapor-permeation separation of a mixture of methanol and other liquid, such as methanol-ethanol, methanol-methyl tert-butyl ether or the like.

In addition, the SAPO-34 molecular sieve membrane of the present invention can also be used for the pervaporation or vapor-permeation separation of a gas-liquid mixture.

### BRIEF DESCRIPTION OF THE DRAWINGS

The invention will be explained in further detail by taking the appended figures and the detailed implementation.

FIG. 1 is an XRD (X-ray diffraction) pattern of the SAPO-34 molecular sieve seeds.

FIG. 2 is an SEM (Scanning Electron Microscopy) image of the SAPO-34 molecular sieve seeds.

FIG. 3 are graphs comparing the surface SEM image of SAPO-34 molecular sieve membrane prepared in Example 1 and the surface SEM image of SAPO-34 molecular sieve membrane prepared via hydrothermal synthesis;
wherein, FIGS. 3A & B are the surface and cross-section images of SAPO-34 molecular sieve membrane prepared by hydrothermal synthesis;
and FIGS. 3 C & D are the surface and cross-section images of SAPO-34 molecular sieve membrane prepared by dry-gel process in Example 1.

FIG. 4A is the surface SEM image of the SAPO-34 molecular sieve membrane prepared in Example 2.

FIG. 4B is the cross-section SEM image of the SAPO-34 molecular sieve membrane prepared in Example 2.

FIG. 5 is a schematic diagram of a pervaporation process, wherein 1 denotes feed liquid, 2 denotes peristaltic pump, 3 denotes molecular sieve membrane assembly and heat source, 4 denotes stop valve, 5 denotes cold trap, 6 denotes vacuum gauge, 7 denotes vacuum pump.

### EXAMPLES

### Example 1

Step 1. 2.46 g of DI water were added into 31.13 g of tetraethylammonium hydroxide solution (TEAOH, 35 wt %), and then 7.65 g of aluminum isopropoxide were added thereto, and the resultant was stirred for 2-3 h at room temperature. Then 1.665 g silica sol (40 wt %) was added dropwise and the resultant was stirred 1 h. Finally, 8.53 g phosphoric acid solution (H₃PO₄, 85 wt %) were slowly added dropwise. The resulting solution was stirred overnight (e.g. stirred for 12 h). Then crystallization is performed at 180° C. for 7 h by using microwave heating. The obtained product was taken out from the reactor, centrifuged, washed, dried, to obtain SAPO-34 molecular sieve seeds.

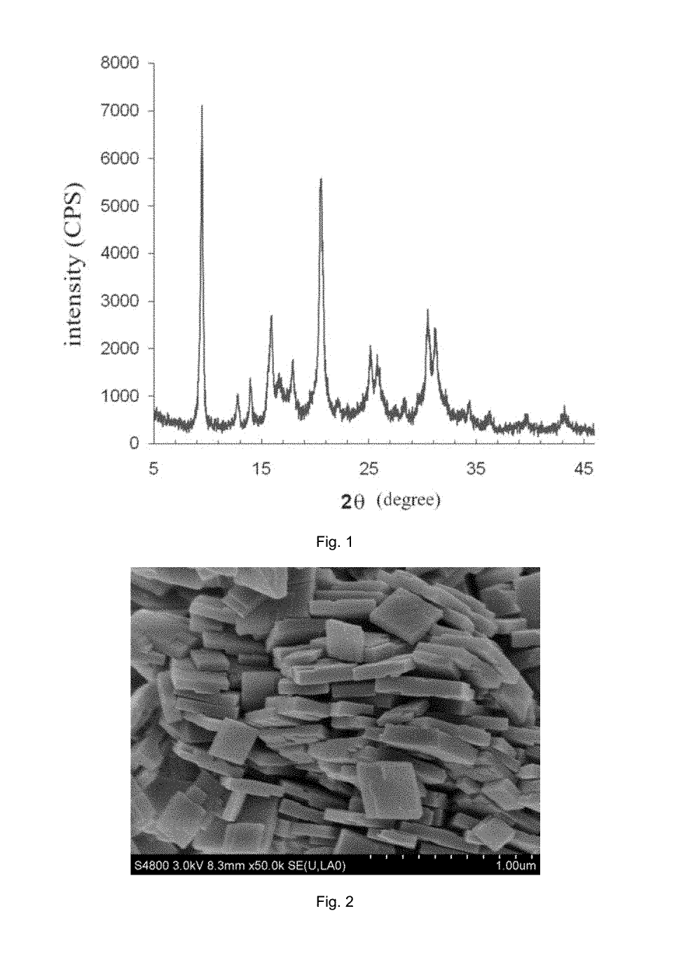

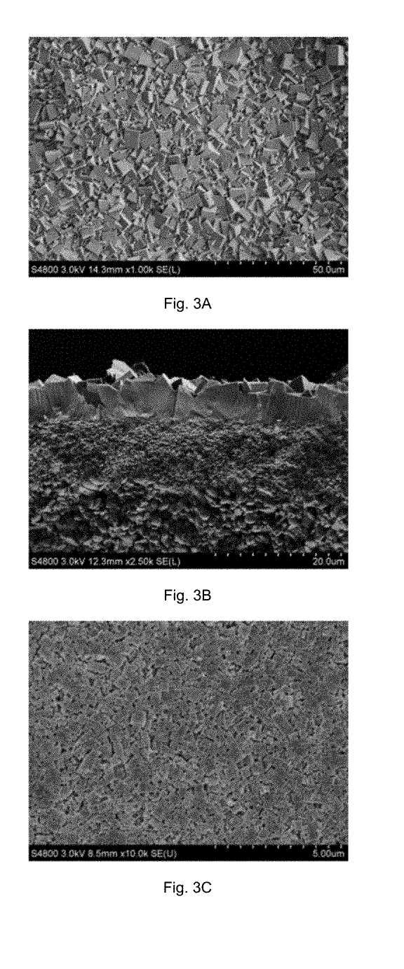

The XRD pattern and the SEM image of the seeds are shown in FIG. 1 and FIG. 2, respectively. Its XRD pattern is consistent with the standard pattern of SAPO-34 molecular sieve, indicating that no mixed crystals were present. It can be seen from FIG. 2 that the size of the seeds is around 300 nm*300 nm*100 nm.

A porous ceramic tube (a porous ceramic tube having an inner diameter of 7 mm, an outer diameter of 10 mm and a length of 600 mm, the shape of the porous support being single-channel tube, and its material being alumina) with 5 nm pore size was used as a support. The two ends of the support were sealed with glaze. After washing and drying, the out surface of the support was sealed (covered) by Teflon tape. Then the SAPO-34 molecular sieve seeds are coated on the inner surface of the porous ceramic tube by brush coating.

Step 2. 9.07 g of aluminium isoproxide were added to 5.12 g of phosphoric acid solution (85 wt %) and 23.33 g of DI water. After full hydrolysis, 1.00 g of silica gel (40 wt %) and 9.34 g of tetraethyl ammonium hydroxide (35 wt %) were added in turn. The resultant was stirred overnight (e.g., stirred for 12 h) to form a mother liquor for dry gel synthesis of molecular sieve membrane.

The resulting mother liquor had a molar composition of 1.0 Al₂O₃: 1.0 P₂O₅: 0.3 SiO₂: 1TEAOH: 77 H₂O.

Step 3. The porous ceramic tube coated with SAPO-34 molecular sieve seeds prepared in step 1 was soaked in the mother liquor for dry gel synthesis for 10 min, taken out and dried at room temperature for 10 min, thereby to have a dry gel layer formed. Then, the porous ceramic tube having a dry gel layer was placed in a 100 mL reaction vessel, and then 1 mL of DI water was added. The dry gel was crystallized for 5 h at 220° C. After washing and drying, a SAPO-34 molecular sieve membrane tube was obtained.

The membrane thickness of the SAPO-34 molecular sieve membrane prepared in Example 1 is around 1 micron (FIGS. 3 C and D). Moreover, the surface of the membrane was completely covered by square lamellar crystals which are well cross-linked therebetween. However, the SAPO-34 molecular sieve membrane prepared by conventional hydrothermal synthesis was covered by cubic crystals which are perfectly crosslinked, and the thickness of the membrane was 5-6 microns (FIGS. 3 A and B). Thus, the thickness of the SAPO-34 molecular sieve membrane prepared in Example 1 is only ⅕ of the thickness of the membrane prepared via hydrothermal method. The mass transfer resistance of a molecular sieve membrane is inversely proportional to its thickness, which means that the flux of the membrane prepared via dry-gel process may be 5 times higher than the membrane prepared by the common hydrothermal process.

Step 4. The SAPO-34 molecular sieve membrane tube obtained in step 3 was calcined under vacuum at 400° C. for removal of the template agent (the temperature increasing rate and the temperature decreasing rate were both 1 K/min), to get a ultrathin SAPO-34 molecular sieve membrane prepared via dry gel process.

Step 5. The ultrathin SAPO-34 molecular sieve membrane prepared in step 4 was used to separate a methanol/dimethyl carbonate (i.e., DMC/MeOH) mixture by a process of pervaporation separation, wherein the feed temperature (i.e., separation operation temperature) is 120° C., the composition of the feed MeOH/DMC is 90/10 (mass ratio), the feed flow rate is 1 mL/min, the system pressure is 0.3 MPa and the permeate side pressure is 100 Pa.

The separation factor is calculated from: α=(w₂ₘ/w_{2d})/(w₁ₘ/w_{1d}), where w₂ₘ is the mass concentration of methanol on the permeate side, w_{2d} is the mass concentration of dimethyl carbonate on the permeate side, w₁ₘ is the mass concentration of methanol in the feed and w_{1d} is the mass concentration of dimethyl carbonate in the feed.

The permeation flux equation is J=Δm/(sxt), where Δm is the mass (unit g) of a product collected on the permeate side, s is the area (m²) of the molecular sieve membrane and t is the collection time (h).

TABLE 1
The vapor-permeation separation test
results of MeOH/DMC in Example 1.

| | Feed | | |
|---|---|---|---|
| Membrane | composition | Permeation fluxJ | Separation |
| tube | MeOH/DMC | [kg/(m² · h)] | factor α |
| Dry-gel | 90/10 | 10.6 | 790 |
| Hydrothermal | 90/10 | 2.1 | 1800 |
| synthesis | | | |

It can be seen from Table 1 that the separation factor for the methanol/dimethyl carbonate of the ultrathin SAPO-34 molecular sieve membrane prepared by dry-gel process is lower than that of the SAPO-34 molecular sieve membrane prepared by hydrothermal synthesis, which was reduced from to 790. But the flux of the former one is increased by 4 times, i.e. from 2.1 kg/(m²·h) to 10.6 kg/(m²·h). It is apparent that the decreasing of the membrane thickness greatly reduces the mass transfer resistance of the membrane, thereby resulting in a great enhancement of the flux. In comparison, although the separation factor of the ultrathin SAPO-34 molecular sieve membrane prepared by dry-gel process decreases to some extent, a separation factor of 790 is high enough because the concentration of methanol in the permeate is higher than 99.9 wt %.

### Example 2

All steps in this Example are the same as in Example 1 except that the porous support coated with SAPO-34 molecular sieve seeds was soaked in the mother liquor for dry gel synthesis for 2 h in step 3.

TABLE 2
The vapor-permeation separation test
results of MeOH/DMC in Example 2.

| Operation | | |
|---|---|---|
| temperature | Permeation flux J | Separation |
| ° C. | [kg/(m² · h)] | factor α |
| 120 | 13.1 | 350 |

It can be seen from Table 2 that in the dry-gel synthesis method, the ultrathin SAPO-34 molecular sieve membrane prepared by immersion in the mother liquor for 2 h has a methanol selectivity of 350, but the flux is higher than 13 kg/(m²·h) (Table 2).

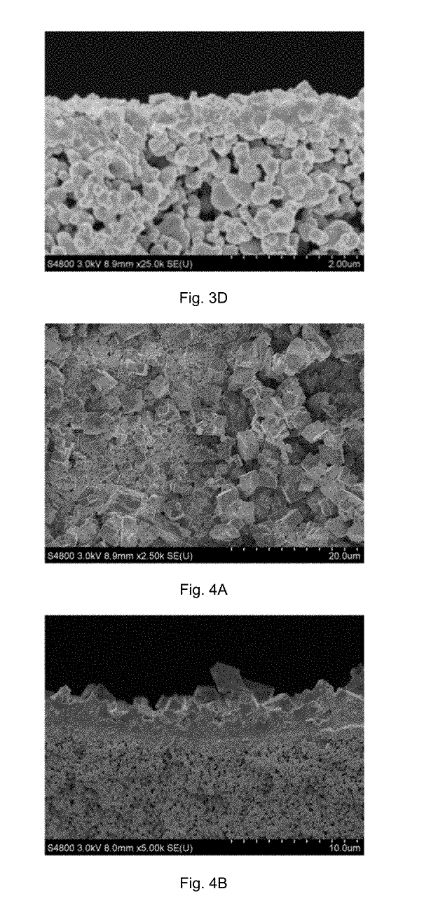

The surface and cross section SEM images of the SAPO-34 molecular sieve membrane prepared in Example 2 are shown in FIGS. 4A and 4B. It can be seen from FIGS. 4A and 4B that the support surface is completely covered by lamellar square crystals which are well crosslinked therebetween. The membrane thickness is relatively uniform, and is about 2-3 microns.

### Example 3

Example 3 differs from Example 1 in that: in step 2), 8.46 g of aluminium hydroxide were added to 10.24 g of phosphoric acid solution (85 wt %) and 31.82 g of DI water. And after adequate hydrolysis, 4.00 g of silica sol (40 wt %) and 33.62 g of tetraethyl ammonium hydroxide (35 wt %) were added in turn. The resultant was stirred overnight (e.g., stirred for 12 h) to form a mother liquor for dry gel synthesis of molecular sieve membrane. The resulting mother liquor had a molar composition of 1 Al₂O₃: 1 P₂O₅: 0.6 SiO₂: 1.8 TEAOH: 77H₂O. Other steps of Example 3 are the same as that of Example 1.

TABLE 3
The vapor permeation separation test
results of MeOH/DMC in Example 3.

| Operation | | |
|---|---|---|
| temperature | Permeation flux J | Separation |
| ° C. | [kg/(m² · h)] | factor α |
| 120 | 12.6 | 128 |

It can be seen from Table 3 that when a dry gel synthesis method is used and the mother liquor has a molar composition of 1 Al₂O₃: 1 P₂O₅: 0.6SiO₂: 1.8TEAOH: 77H₂O, the synthesized ultrathin SAPO-34 molecular sieve membrane has a methanol selectivity of 128 and a flux higher than 12 kg/(m²·h).

### Example 4

All steps in this Example are the same as in Example 1 except that in step 3), the porous support coated with SAPO-34 molecular sieve seeds was soaked in the mother liquor for dry gel synthesis for 40 min and 2 mL DI water was added to the 100 mlLreaction vessel.

TABLE 4
The vapor permeation separation test
results of MeOH/DMC in Example 4.

| Operation | | |
|---|---|---|
| temperature | Permeation flux J | Separation |
| ° C. | [kg/(m² · h)] | factor α |
| 120 | 12.9 | 305 |

It can be seen from Table 4 that when a dry gel synthesis method is used and 2 g of DI water were added to the bottom of the 100 mL hydrothermal reaction vessel, the synthesized ultrathin SAPO-34 molecular sieve membrane had a methanol selectivity of 305 and a flux higher than 12 kg/(m²·h).

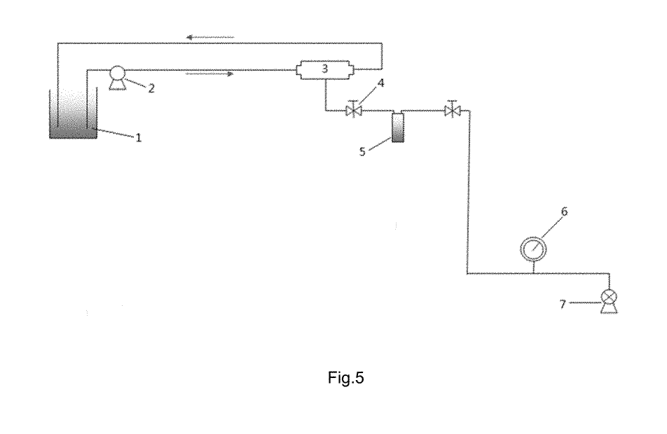

In addition, the pervaporation process of Examples 1-4 is shown in FIG. 5.

In addition, the SAPO-34 molecular sieve membranes prepared as above can also be used for the pervaporation or vapor-permeation separation of a gas-liquid mixture, wherein the gas of the gas-liquid mixture may be one of nitrogen gas, hydrogen gas, oxygen gas, carbon dioxide or methane or the like; and the liquid of the gas-liquid mixture may be one of water, methanol, acetone or benzene or the like.

1. A method for the pervaporation or vapor-permeation separation of a gas-liquid mixture or a liquid mixture by a SAPO-34 molecular sieve membrane prepared in a dry gel process, characterized in that the method comprises the following steps;
1) mixing and dissolving an Al source, tetraethylammonium hydroxide (TEAOH), water, a Si source and a P source to make a reaction liquor for seeds, which is then subjected to crystallization for 2-72 h by heating at 120-230 ±C, then centrifuging, washing and drying to get SAPO-34 molecular sieve seeds;
wherein the molar ratio of the Al source, P source, Si source, tetraethylammonium hydroxide and all water in the reaction liquor for seeds is 1 Al₂O₃: 1-2 P₂O₅: 0.3-0.6 SiO₂: 1-3 TEAOH: 55-150 H₂O;
2) coating the SAPO-34 molecular sieve seeds on a porous ceramic support;
3) uniformly mixing an Al source, a P source, a Si source, tetraethyl ammonium hydroxide (TEAOH) and water to form a mother liquor for dry gel synthesis of SAPO-34 molecular sieve membrane;
wherein the molar ratio of the Al source, P source, Si source, tetraethyl ammonium hydroxide and all water in the mother liquor for dry gel synthesis is: 1 Al₂O₃: 1-2 P₂O₅: 0.1-0.6 SiO₂: 1-8 TEAOH:30-1000 H₂O;
4) supporting the mother liquor for dry gel synthesis on the porous support coated with SAPO-34 molecular sieve seeds prepared in step 2), and drying, to form a porous support having a dry gel layer;
5) placing the porous support prepared in step 4) into a reaction vessel, adding a solvent, performing crystallization of the dry gel;
6) calcining at 400-600° C. for 4-8 h to obtain a SAPO-34 molecular sieve membrane;
7) using the SAPO-34 molecular sieve membrane obtained in step 6) to perform the separation of a liquid mixture by a process of pervaporation separation or vapor-permeation separation;
wherein the liquid mixtures is a mixture of methanol and a liquid other than methanol, wherein said liquid other than methanol is selected from one of dimethyl carbonate, ethanol, methyl tert-butyl ether;

2. The method according to claim 1 characterized in that in step 1), the procedure for making the reaction liquor for seeds comprises adding the Al source to the tetraethyl ammonium hydroxide solution, and after hydrolysis, adding the Si source and the P source, and stirring for 12-24 h to form the reaction liquor for seeds; wherein the heating is microwave heating; and the size of the SAPO-34 molecular sieve seeds is 50-1000 nm.

3. The method according to claim 1 characterized in that in steps 1) and 3), the Al source is selected from one or more of aluminium isopropoxide, aluminium hydroxide, elemental aluminium, an aluminium salt, alumina, and hydrated alumina; wherein the aluminium salt is selected from one or more of aluminium nitrate, aluminium chloride, alumimium sulfate, aluminium phosphate;
the Si source is selected from one or more of silica sol, silicate ester, silica aerosol and sodium silicate;
the P source is phosphoric acid.

4. The method according to claim 1 characterized in that in step 2), the shapes of the porous support are selected from single-channel tube, multi-channel tube, flat plate, hollow fiber tube; the pore size of the porous support is 5-2000 nm; and the coating method is selected from brush coating, dip-coating, spray coating or spin coating.

5. The method according to claim 4 characterized in that the coating method is dip-coating and the process comprises uniformly coating a 0.01-1 wt % solution of the SAPO-34 molecular sieve seeds in ethanol on the porous support.

6. The method according to claim 1 characterized in that in step 3), the procedure of forming the mother liquor for dry gel synthesis of SAPO-34 molecular sieve membrane comprises adding the Al source to the P source and after full hydrolysis, adding the Si source and tetra-ethyl ammonium hydroxide and stirring for 12-24 h, to obtain the mother liquor for dry gel synthesis of SAPO-34 molecular sieve membrane.

7. The method according to claim 1 characterized in that in step 4), the supporting method is selected from dip-coating, spray coating or spin coating; and the molar composition of the dry gel layer is 1.0 Al₂O₃: 1-2.0 P₂O₅: 0.1-0.6 SiO₂: 1-8 TEAOH: 1-300 H₂O.

8. The method according to claim 7 characterized in that the supporting method is dip-coating and its procedure comprises immersing the porous support coated with SAPO-34 molecular sieve seeds in the mother liquor for dry gel synthesis for 1 s to 24 h, and then drying for 1 min to 48 h at room temperature to 120° C.

9. The method according to claim 1 characterized in that in step 5), the solvent is selected from one or more of water, ammonia water, the mother liquor for dry gel synthesis, an organic solvent; and the solvent is used in an amount of 0.001˜0.1 g per mL volume of the reaction vessel; and the temperature for crystallization of the dry gel is 120-230° C. and the crystallization time is 2-72 h.

10. The method according to claim 9 characterized in that the crystallization time is 4-7 h.

11. The method according to claim 1 characterized in that in step 6), the atmosphere for calcination is selected from inert gas, vacuum, air, oxygen gas, or diluted oxygen gas in any ratio; in the calcination, the temperature increasing rate and the temperature decreasing rate are not higher than 2 K/min; and the membrane thickness of the SAPO-34 molecular sieve membrane is 1-2 microns.

12. The method according to claim 1 characterized in that in step 7), the conditions for the process of pervaporation separation or vapor-permeation separation comprises: a methanol concentration of the feed ranging from 1-99 wt %, a feed flow rate ranging from 1-500 mL/min, a separation operation temperature ranging from 20° C. to 150° C., and a pressure on the permeate side ranging from 0.06 Pa to 300 Pa;
wherein in step 7), the inert gas contains N₂;
the gaseous hydrocarbon contains methane;
the alcohol contains methanol, ethanol, or propanol;
the ketone contains acetone or butanone;
the aromatics contain benzene.
